(19) United States
(12) Patent Application Publication
(10) Pub. No.: US 2019/0000324 A1
(43) Pub. Date: Jan. 3, 2019

(54) BANDAGE MEMBER

(71) Applicant: C-Patient ApS, Soenderborg (DK)

(72) Inventor: Steffan LAUGAARD NIELSEN, Augustenborg (DK)

(21) Appl. No.: 16/047,982

(22) Filed: Jul. 27, 2018

Related U.S. Application Data
(63) Continuation of application No. PCT/DK2017/050016, filed on Jan. 26, 2017.

(30) Foreign Application Priority Data
Jan. 28, 2016 (DK) PA 2016 00055

(51) Int. Cl. A61B 5/01; A61B 5/00

(52) U.S. Cl. CPC A61B 5/01; A61F 13/00004; A61B 5/6833; A61B 5/0008

(57) ABSTRACT

A bandage sensory member for monitoring temperature differences is disclosed. The bandage sensory member comprises a sensory layer having a plurality of temperature sensors disposed thereon, and a chip unit directly or indirectly electrically connected to the temperature sensors. The chip unit is configured to detect the temperature of each of the temperature sensors and send a wireless signal to an external receiver. The wireless signal contains information about the temperature difference and/or maximum temperature difference between the temperature sensors.

### CROSS-REFERENCE TO RELATED APPLICATIONS

This application is a continuation of International Application No. PCT/DK2017/050016, filed Jan. 26, 2017, which claims priority to Danish Patent Application No. PA 2016 00055, filed Jan. 28, 2016, the entire contents of which are hereby incorporated by reference into this application.

### FIELD OF INVENTION

The present invention relates to a bandage sensory member useful for tissue monitoring. The bandage sensory member of the present invention is suitably incorporated into a bandage for application to the skin of a patient. Thereby, the bandage sensory member allows the patient to monitor tissue in an easy manner.

### PRIOR ART

It is well known that temperature differences indicate infection in a wound. Therefore, detection of the temperature difference is a commonly used tool in tissue monitoring. Several attempts have been made to provide monitor structures capable of monitoring the temperature of wounded tissue in order to detect wound complications such as infection. Most of the prior art solutions are, however, expensive, and complicated.

From US 2009/0204100 A1, a body cover comprising temperature sensing elements is known. The temperature sensing elements detects the temperature of proximate skin and converts the local temperature into a visual and/or electrical signal. However, it is essential the temperature sensing elements are able to measure the temperature with high accuracy, thereby making the body cover rather expensive.

Accordingly, there is a need for an alternative monitor structure which reduces or even eliminates the above-mentioned disadvantages of the prior art.

It is an object of the present invention to provide a simple and cost-effective bandage sensory member for monitoring of wound complications such as infection.

### SUMMARY OF THE INVENTION

The object of the present invention can be achieved by a bandage sensory member as defined in claim 1. Preferred embodiments are defined in the dependent sub claims, explained in the following description, and illustrated in the accompanying drawings.

In a first aspect, the present invention relates to a bandage sensory member comprising a sensory layer having a plurality of temperature sensors disposed thereon, and a chip unit directly or indirectly electrically connected to the temperature sensors, wherein the chip unit is configured to detect the temperature of each of the temperature sensors and send a wireless signal to an external receiver, wherein the wireless signal contains information about the temperature difference (ΔT) and/or the maximum temperature difference (ΔTₘₐₓ) between the temperature sensors.

In another aspect, the present invention relates to a bandage sensory member comprising a sensory layer having a plurality of temperature sensors disposed thereon, and a chip unit directly or indirectly electrically connected to the temperature sensors, wherein the chip unit is configured to detect the temperature of each of the temperature sensors and send a wireless signal to an external receiver, wherein the wireless signal contains information about the maximum temperature difference (ΔTₘₐₓ) between the temperature sensors.

In a second aspect, the present invention relates to bandage comprising a bandage sensory member according to the present invention.

The sensors of the bandage sensory member are configured to monitor local temperature differences and the chip unit provides information about the temperature difference and/or the maximum temperature difference between each of the sensors in the sensory layer. In some embodiments, it is desirable to obtain information about the temperature difference between the sensors. In other embodiments, it is desirable to obtain information about the maximum temperature difference between the sensors. In further embodiments, it is desirable to obtain information about the temperature difference as well as the maximum temperature difference between the sensors.

Thus, a simple and cost effective bandage sensory member for monitoring the condition or state of a skin lesion can be obtained. Skin lesions may be the result of surgical procedures like operation, or wounds in general. In particular, the bandage sensory member of the invention provides valuable information as regards possible complications such as signs of infection or inflammation. The expressions “wounded tissue” and “skin lesion” is used interchangeably herein.

In certain embodiments, the bandage sensory member may comprise one or more additional layers. One or more additional layers may be provided on each side of the sensory layer, or only on the one side of the sensory layer. One or more additional layers may further, in some embodiments, be provided on both sides of the sensory layer. The sensory layer and the additional layer or additional layers may be joined together or fastened by using an adhesive or by heat or steam treatment. Suitable adhesives are well-known in the art and may be provided on the sensory layer, or on the additional layer(s), or on both the sensory layer and the additional layer(s). The additional layer may in some embodiments be a material that encapsulates the sensory layer so that the bandage sensory member has a planar surface. This may in particular be suited in case of pressure-sensitive wounds or lesions.

The temperature sensors applied are suitably arranged in such a manner that the temperature of the wounded tissue and the temperature of the non-wounded tissue are detected at the same time, i.e. simultaneously. Accordingly, the bandage sensory member needs not be arranged or applied in any specific manner as long as temperature sensors are positioned so as to measure the temperature of at least a part of both the wounded tissue and the non-wounded tissue (at least one sensor for wounded issue and at least sensor for non-wounded tissue). Thus, as long as the temperature of the wounded tissue and the non-wounded tissue are detected, the bandage sensory member will function. It may, however, be preferred that temperature sensors are positioned so as to sense the temperature of a representative selection of the whole wounded tissue area as well as a sufficient area of non-wounded tissue. Thereby, reliable information of the temperature difference and/or the maximum temperature difference can be obtained. In a specific embodiment, the temperature sensors are disposed onto the sensory layer in a matrix configuration, ensuring that the sensors provide information about a representative selection of both wounded and non-wounded tissue.

The bandage sensory member may have any suitable size and geometry, like e.g. square shaped, rectangularly shaped, circular-shape, curved, irregularly shaped. Accordingly, it is possible to provide a large bandage sensory member to cover a large area of tissue. Likewise, it is possible to provide a smaller bandage sensory member to cover a smaller area of tissue.

It may be an advantage that the bandage sensory member is configured to be bent along the contours of the body, particularly the human body, including the extremities. Thus, the bandage sensory member may be shaped to fit certain body parts or body areas.

The bandage sensory member comprises a chip unit electrically connected to the temperature sensors and configured to detect the largest (maximum) temperature difference between any of the temperature sensors. In some embodiments, the chip unit merely detects the temperatures measured by the temperature sensors. Hereby, a device receiving the detected temperatures can automatically determine if any temperature difference exceeds a predefined level indicative of infection.

The chip unit is configured to send a wireless signal to an external receiver (e.g. a smartphone, a tablet, a reader, or another electronic device) by means of an antenna. Accordingly, only a simple, short-range transmitter is required. Therefore, it is possible to provide a simple and cost-efficient bandage member.

The wireless signal sent to the external receiver (e.g. a smartphone, a tablet, a reader, or another electronic device) may contain information including the temperature difference and/or the maximum temperature difference between the temperature sensors. The wireless signal sent to the external receiver (e.g. a smartphone) may contain merely the temperatures detected by the temperature sensors. Accordingly, the external receiver (e.g. a smartphone, a tablet, a reader, or another electronic device) can forward this information, e.g. with the purpose of generating an alert, to specific receivers (e.g. health care personnel).

It may be an advantage that a coil member is integrated in the bandage sensory member, wherein the coil member is configured to generate electrical power by means of induction upon moving the external receiver (e.g. a smartphone, a tablet, a reader, or another electronic device) above the bandage sensory member. Hereby, the bandage sensory member needs no battery, and the energy for detecting, processing and sending temperature signals can be harvested by means of the coil member.

It is an advantage that the coil member is configured to send a wireless signal to an external receiver. Hereby, the bandage sensory member does not need to comprise a sending unit capable of sending long-range signals.

It may be advantageous that the bandage sensory member comprises a temperature sensor arranged and configured to detect the ambient temperature. It is to be understood that more than one ambient temperature sensor may be used. This is e.g. suitable to lower the risk of malfunction of the ambient temperature sensor. The actual number of ambient temperature sensors may depend on the size and geometry of the bandage sensory member, but from 1-10, such as from 1-5, in particular 1, 2 or 3 sensors may be suitable and sufficient. It is to be understood that the ambient temperature sensors may be disposed onto the sensory layer and/or onto an additional layer.

The bandage sensory member may use the ambient temperature to compensate for the ambient temperature. If the ambient temperature is low (e.g. lower than 15° C.), cooling of the bandage sensory member may be expected, and thus lower temperature measurements of the temperature sensors may be achieved.

However, if the ambient temperature is high (e.g. above 30° C.), heating of the bandage sensory member may be expected, and thus higher temperature measurements of the temperature sensors may be achieved.

By detecting the ambient temperature, it is possible to adjust for the ambient temperature so that the temperatures detected by the temperature sensors are adjusted according to the ambient temperature. The external receiver (e.g. smartphone, a tablet, a reader, or another electronic device) may be configured to adjust the temperatures detected by the temperature sensors on the basis of the detected ambient temperature.

The chip unit may be configured to adjust the temperatures detected by the temperature sensors on the basis of the detected ambient temperature.

It may be beneficial that the bandage sensory member comprises a storage unit, preferably a storage unit provided in the chip unit.

Hereby, the storage unit makes it possible to store data and send the data later.

It may be advantageous that the bandage sensory member comprises a near field communication (NFC) unit, preferably a NFC unit configured to communicate with a smartphone by using NFC. It may be beneficial that the bandage sensory member comprises a NFC unit integrated in a chip unit, wherein an adapter/controller is electrically connected to the chip unit and adapted to receive a plurality of electrical connections and combine electrical signals from these different electrical connections to a single entry of the chip unit.

It may be an advantage that the chip unit is a radio-frequency identification (RFID) chip unit, wherein an adapter/controller is electrically connected to the RFID chip unit and adapted to receive a plurality of electrical connections and combine electrical signals from these different electrical connections to a single entry of the RFID chip unit.

Hereby, it is possible to apply a simple (and low-cost) chip unit having only a single electrical entry. The chip unit and the adapter/controller are electrically connected to the wire member and the coil member. A RFID chip uses wireless electromagnetic fields to transfer data, for the purposes of automatically identifying and tracking tags.

It may be advantageous that the chip unit is configured to generate an alert:
in case no scanning of the temperature sensors has been conducted within a predefined time period (e.g. two or eight hours) and/or
in case the maximum temperature difference (ΔTₘₐₓ) exceeds a predefined value.

Hereby, the bandage sensory member can be used to monitor wounded tissue in patients in a safe and reliable manner.

It may be beneficial that the coil member surrounds the temperature sensors.

Hereby, the most efficient antenna can be achieved. Moreover, it is easier to establish an inductive coupling between the coil member and a smartphone or another external receiver when a large coil used. Furthermore, more energy can be generated by using a large coil.

It may be an advantage that all temperature sensors are electrically connected. Hereby, all detected temperatures can be received by one receiving member (the chip unit or the adapter/controller electrically connected to the chip unit).

It may be an advantage that all temperature sensors are electrically connected by means of a wire member. The wire member may comprise one or more conductors.

It may be advantageous that all temperature sensors are arranged in a matrix configuration. In some embodiments, the temperature sensors are arranged in a certain pattern. E.g. there may be more sensors on the area intended for application onto the wounded tissues area, or more sensors on the area intended for application onto the non-wounded area.

The bandage sensory member of the invention can be produced in an easy manner, and user-friendliness is increased since it is possible to provide a symmetric bandage member.

It may be beneficial that the matrix configuration comprises nine or twelve or sixteen temperature sensors. However, any number of temperature sensors may be present. In general, the number of temperature sensors may suitably be from 2-24, such as 2, 3, 6, 9, 12, 15, 18, 21 or 24. In some embodiments, it may be sufficient to use only 2 sensors, whereby one sensor detects the temperature of the non-wounded tissue, and one sensor detects the temperature of the wounded tissue. In addition, ambient temperature sensors as described above may be present.

It is possible to apply a bandage sensory member having a chip unit that comprises an energy storage. Hereby, it is possible to perform temperature measurements and send information from the bandage sensory member even when no external receiver (e.g. a smartphone, a tablet, a receiver, or another electronic device) is in the proximity of the bandage sensory member.

It may be beneficial that the sensory layer, the additional layer(s) and/or the bandage sensory member is (are) provided with a plurality of apertures. Hereby, the apertures can ensure that sufficient ventilation is provided to a wound and help absorb fluids from the wound, when the bandage sensory member is incorporated into a bandage used to monitor a wound.

The construction of bandages and bandaids are generally well-known in the art. However, when using the bandage sensory member of the invention for providing a bandage or a bandaid, it may be an advantage that at least a portion of the bandage sensory member is coated. Hereby, it is possible to prevent fluid (e.g. blood and water) from accessing the inner structure of the bandage sensory member, including the wire member and the temperature sensors. In some embodiments, the bandage sensory member is coated with one or more coating layers (i.e. an additional layer).

It may be advantageous that the bandage sensory member comprises an absorbing layer (example of an additional layer). The absorbing layer is capable of absorbing excess fluid. It may be an advantage that the absorbing layer is provided to fit apertures of the bandage or bandaid.

The bandage or bandaid as well as the layers of the bandage sensory member may be made of any suitable material used to make bandages. The material may be a woven or non-woven material. Such materials are generally known in the art.

In some embodiments, the sensory layer of the bandage sensory member may be one or more of a plate, a foil, a plastic material, or a paper material, optionally provided with an adhesive on the one side or on both sides. Furthermore, the sensory layer may be provided with apertures to minimise the surface area, to ensure flexibility of the sensory layer or to allow other materials be present (e.g. a foam, a textile layer, or an absorbing layer).

In some embodiment, the additional layer of the bandage sensory member may be one or more of a foam, a textile, a coating, a plastic material, or a polymeric material.

It may be an advantage that the wire member and coil member are flat.

It may be an advantage that indications are provided at the surface of the bandage sensory member. Accordingly, the indications (e.g. dotted line) can indicate the positions in which the temperature sensors are provided.

The temperature sensors to be used in the bandage sensory member of the invention may be any type suitable for the purpose of the invention. In some embodiments, analogue sensors may be used. The temperature sensors and the chip unit are calibrated prior to or subsequently to being disposed onto the sensory layer. In a preferred embodiment, the temperature sensors and the chip unit are calibrated after being disposed onto the sensory layer. In another preferred embodiment, the temperature sensors and the chip unit are calibrated, after the bandage sensory member is formed. Calibration may suitably be performed by a process comprising placing the temperature sensors and the chip unit (when disposed on the sensory layer, or when the bandage sensory member is formed) in surroundings having a constant temperature for a sufficient period of time, followed by setting a “zero” temperature point (“zero” point) for the temperature sensors and the chip unit using a suitable application. By “surroundings” is meant a place where the temperature can be held constant. Examples include a water bath and a chamber. By “sufficient period of time” is meant the period of time necessary to ensure all temperature sensors and the chip unit have reached the same temperature. Examples of sufficient periods of time are from seconds to minutes or hours, e.g. 5 seconds to 5 minutes. By “suitable application” is meant a program, an app or other software which is able to designate the “zero” temperature value to the temperature sensors and the chip unit. Such applications are commercially available. It is to be understood that the temperature need not be 0° C., but can be any temperature. Examples of suitable “zero” temperatures include 2° C., 4° C., 6° C., 8° C., and 10° C. as well as any non-integer therebetween.

The above-mentioned calibration is an important aspect of the present invention as it allows for the use of temperature sensors that does not measure the precise and actual temperature. In accordance with the present invention, it is sufficient to know the temperature difference between the sensors, preferably the maximum temperature difference, and this is obtained by setting the arbitrary “zero” temperature. This is advantageous compared to using temperature sensors which are capable of measuring the exact temperature of the surroundings, e.g. the skin, and allows for the use of inexpensive and rather imprecise temperature sensors.

The bandage sensory member may suitably be integrated in a commercially available bandage or bandaid and be used in connection with treatment of lesions, wounds, or sores, and following surgery, as well as a precautionary means for monitoring possible inflammatory conditions. However, the bandage sensory member of the invention may also be used alone to monitor lesions, wounds, or sores.

In general, a temperature difference of from 2-10° C., such as e.g. 1° C., 2° C., 3° C., 4° C., or 5° C. as any non-integer therebetween, is indicative of inflammation or infection. In particular, a temperature difference between 2° C. and 3° C., such as between 2.1° C. and 2.5° C., may be indicative of inflammation or infection. More specifically, a temperature difference of 2.2° C. may be indicative of inflammation or infection. The temperature difference may be measured as the temperature difference between the sensors or the maximum temperature difference between the sensors.

In particular, the bandage sensory member may be applied as part of a bandage in the case of diabetic ulcers, such as diabetic foot ulcers. Diabetic foot ulcers are a common and frequent risk as a result of the diabetic condition. The prevalence of diabetes is increasing and the onset often occur at a younger age. Accordingly, serious complications are increasing and a major issue. All too often, diabetic ulcers lead to amputation of the foot or even the leg, and often results in recurring diabetic ulcers and an increased mortality. Over time, the diabetic patient experience loss of feeling or numbness due to nerve damage, which typically affects hands, arms, feet, and legs. People who cannot sense pressure from a pinprick or monofilament have lost protective sensation and are at risk for developing foot ulcers that may not heal properly. Furthermore, sores or injuries may not be noticed and may become ulcerated or infected. Circulation problems also increase the risk of foot ulcers. Likewise, temperature perception may also be affected and it may be difficult to sense if an infection is under development.

Thus, in another aspect, the present invention relates to the use of a bandage sensory member according to the present invention incorporated into a bandage or bandaid for monitoring the temperature of a wound or lesion of the skin. Thereby, the temperature difference, in particular the maximum temperature difference, between areas of the skin can be determined and correlated with infection or inflammation, possibly before the patient notices the condition himself. Accordingly, adequate treatment may be initiated earlier, reducing the risk of serious complications and even amputation of body parts, the latter in particular in case of diabetic ulcers.

### DESCRIPTION OF THE DRAWINGS

The invention will become more fully understood from the detailed description given herein below. The accompanying drawings are given by way of illustration only, and thus, they are not limitative of the present invention. In the accompanying drawings:

FIG. 1 A shows a schematic top view of a sensory layer of a bandage sensory member according to the invention;

FIG. 1 B shows a schematic top view of a sensory layer of a bandage sensory member according to the invention;

FIG. 1 C shows a schematic top view of a sensory layer of a bandage sensory member according to the invention;

FIG. 1 D shows a schematic top view of a sensory layer of a bandage sensory member according to the invention;

FIG. 2 A shows a schematic top view of a sensory layer of a bandage sensory member according to the invention;

FIG. 2 B shows a cross-sectional view of the sensory layer with an additional layer of the bandage sensory member shown in FIG. 2(A);

FIG. 2 C shows a perspective view of a sensory layer with an additional layer of a bandage sensory member according to the invention;

FIG. 2 D shows a close-up view of a portion of the sensory layer with additional layer of the bandage member shown in FIG. 2(B);

FIG. 3 A shows a schematic top view of a bandage sensory member according to the invention;

FIG. 3 B shows a bottom view of the sensory layer of the bandage sensory member shown in FIG. 3(A);

FIG. 3 C shows a perspective bottom view of the sensory layer bandage member shown in FIG. 3(A) and in FIG. 3(B);

FIG. 3 D shows a cross-sectional view of the bandage sensory member shown FIG. 3(A-C);

FIG. 4 A shows a perspective view of a patient wearing a bandage with a bandage sensory member according to the invention and a health care person activating the sensors of the bandage sensory member by means of a smartphone;

FIG. 4 B shows a cross-sectional view of wounded tissue of a patient;

FIG. 4 C shows a top view of a sensory layer of a bandage sensory member according to an embodiment of the invention;

FIG. 4 D shows a bandage sensory member according to another embodiment of the invention;

FIG. 5 A shows an exploded view of the bandage sensory member shown in FIG. 2(B);

FIG. 5 B shows a close-up view of a sensor of the bandage sensory member shown in FIG. 5(A);

FIG. 5 C shows a schematic view of a bandage sensory member according to the invention covering wounded tissue;

FIG. 6 A shows a cross-sectional view of a bandage sensory member according to the invention;

FIG. 6 B shows an exploded view of the bandage sensory member shown in FIG. 6(A) and

FIG. 6 C shows a close-up view of a sensor of the bandage sensory member shown in FIG. 6(A) and FIG. 6(B).

FIG. 7 A shows the temperature curve of the prior art temperature sensors capable of measuring precise temperatures (relative to an absolute “zero” temperature).

FIG. 7 B shows the arbitrary temperature calibration of the temperature sensors applied in accordance with the present invention.

### DETAILED DESCRIPTION OF THE INVENTION

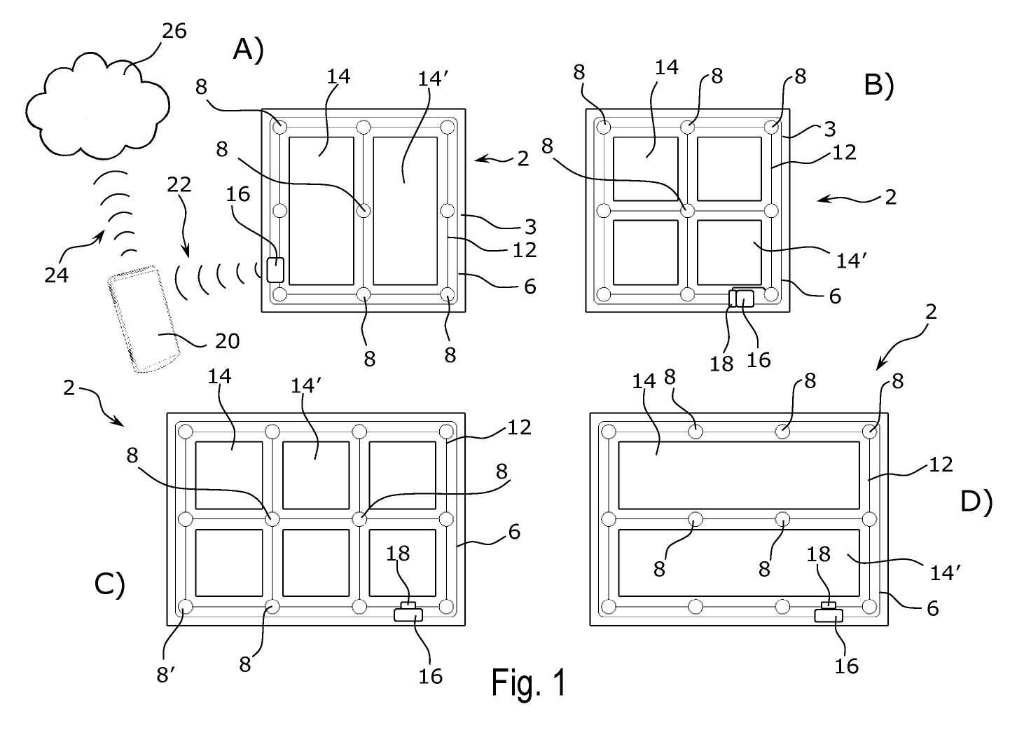

Referring now in detail to the drawings for the purpose of illustrating preferred embodiments of the present invention, a bandage member 2 of the present invention is illustrated in FIG. 1(A). Only the sensory layer 3 is shown. Any additional layers 4 are not shown. The bandage sensory member 2 comprises a sensory layer 3 provided with a plurality of first temperature sensors 8 arranged and configured to detect the reference temperature of non-wounded tissue. The sensory layer 3 additionally comprises an additional temperature sensor 8 arranged and configured to detect the temperature of wounded tissue.

In principle, any of the temperature sensors 8 may measure the reference temperature of non-wounded tissue. Likewise, any of the temperature sensors 8 may measure the reference temperature of a wounded tissue.

A first aperture 14 and a second aperture 14′ are provided in the bandage sensory member 2. The apertures 14, 14′ ensure sufficient ventilation to a wound in case a resulting bandage 5 is used to monitor a wound.

The bandage sensory member 2 comprises a chip unit 16 in the sensory layer 3 electrically connected to the temperature sensors 8 by means of a wire member 12.

The chip unit 16 is electrically connected to a coil member 6 surrounding the temperature sensors 8. The coil member 6 is configured to generate and deliver electrical energy to the chip unit 16 by means of induction when an external receiver formed as a smartphone 20 is brought in proximity to the bandage sensory member 2 or a resulting bandage 5 (the latter not shown).

When a smartphone 20 is brought in proximity to the bandage sensory member 2 or a resulting bandage 5, electrical power is generated in the coil member 6, and this electric power is transferred to the chip unit 16 through the coil member 6. The chip unit 16 comprises a processor that reads the temperatures of the temperature sensors 8 and/or the temperature difference between any of temperature sensors 8. Accordingly, the maximum temperature difference ΔTₘₐₓ is detected either by the chip unit 16 or by an external receiver 20 (e.g. a smartphone).

ΔTₘₐₓ=max{Tᵢ−Tⱼ}, where Tᵢ and Tⱼ is the temperature of the i^{th} the and the j^{th} sensor, respectively. (1)

In one embodiment of the invention, the chip unit 16 is configured to send the detected ΔTₘₐₓ to the smartphone 20 as a wireless signal 22. In another embodiment of the invention, the chip unit 16 is configured to send the detected temperatures T₁, T₂, . . . , T_{N} (where N is the number of temperature sensors) to the smartphone 20 as a wireless signal 22.

The smartphone 20 forwards the information to the internet 26 as a wireless signal 24. Hereby, the smartphone 20 is capable of alerting relevant people (healthcare persons or relatives) in case a special treatment is needed.

In one embodiment of the invention, the smartphone 20 is configured to send an alert in case no scanning of the temperature sensors 8 has been conducted within a predefined time period (e.g. two or eight hours).

The bandage sensory member 2 shown in FIG. 1(A) comprises nine temperature sensors 8; however, the number N of temperature sensors 8 may be varied. It is important though that the bandage sensory member 2 comprises at least one temperature sensor 8 adapted to be arranged near wounded tissue, and that the bandage sensory member 2 comprises at least one temperature sensor 8 adapted to be arranged in non-wounded tissue, so that the difference between the wounded tissue and the non-wounded tissue can be detected.

In one embodiment of the bandage sensory member 2 according to the invention, the bandage sensory member 2 is configured to detect the largest temperature difference ΔTₘₐₓ between any of the temperature sensors 8 and to send a wireless signal 22 when the ΔTₘₐₓ exceeds a predefined value.

In another preferred embodiment of the bandage sensory member 2 according to the invention, the bandage sensory member 2 is configured to send all detected temperatures measured by the temperature sensors 8 as a wireless signal 22 to the smartphone 20. Accordingly, the smartphone 20 may be configured to calculate the largest temperature difference ΔTₘₐₓ between any of the temperature sensors 8 and to determine if ΔTₘₐₓ exceeds a predefined value.

The predefined level may preferably be within the range of from 2.1° C. to 2.5° C. In a preferred embodiment, the predefined level can be changed in either the chip unit 16, the smartphone 20 or in a database. It is preferred that the predefined level can be changed in the smartphone 20 or in a database.

It may be beneficial that the bandage sensory member 2 is configured to generate an alert in case the ΔTₘₐₓ exceeds a predefined value.

It may be a major advantage that the smartphone 20 comprises software or an application enabling the smartphone 20 to generate an alert in case the ΔTₘₐₓ exceeds a predefined value. Hereby, it is possibly to apply a simple and cheap chip unit 16 that is only capable of detecting the temperatures measured by the temperature sensors 8 and sending these temperatures to the smartphone 20. The smartphone 20 may preferably be configured to calculate the largest temperature difference ΔTₘₐₓ between any of the temperature sensors 8 and determine if ΔTₘₐₓ exceeds a predefined value.

In one embodiment, the smartphone 20 comprises a near field communication (NFC) device adapted to communicate by using a short-range, low-power communications protocol between the smartphone 20 and the bandage sensory member 2. The smartphone 20 uses magnetic induction to create a radio-wave field that the coil member 6 of the bandage sensory member 2 can detect and access, allowing small amounts of data to be transferred wirelessly. The bandage sensory member 2 may have a unique identification. The smartphone 20 may be configured to read the unique identification of the bandage sensory member 2 together with the sensor temperature differences {Tᵢ−Tⱼ}, where Tᵢ and Tⱼ are the temperatures of the i^{th} and the j^{th} temperature sensors, respectively.

By using a bandage sensory member 2 according to the invention incorporated into a bandage 5, it is possible to monitor a wound in a simple and reliable manner. In fact, the patient may conduct the monitoring himself by using a smartphone 20. No battery is needed in the bandage sensory member 2. Accordingly, a low-priced bandage sensory member 2 can be achieved.

FIG. 1(B) illustrates a schematic top view of a sensory layer 3 of a bandage sensory member 2 according to the invention. Additional layers 4 are not shown. The bandage sensory member 2 comprises a sensory layer 3 comprising eight peripherally-arranged temperature sensors 8 arranged along the periphery of the sensory layer 3. The eight peripheral temperature sensors 8 are electrically connected by an electric wire member 12 and surrounded by a coil member 6.

A centrally arranged temperature sensor 8 (at the centre of the sensory layer) is electrically connected to the remaining temperature sensors 8 by the wire member 12. The centrally arranged temperature sensor 8 is configured to be arranged to measure the temperature of wounded tissue (or non-wounded tissue), while the remaining eight peripheral temperature sensors 8 are configured to detect the temperature in non-wounded tissue surrounding wounded tissue (or alternatively wounded tissue).

An adapter/controller 18 is electrically connected to the chip unit 16. The adapter/controller 18 makes it possible to connect a plurality of electrical connections to the adapter/controller 18 and combine electrical signals from different electrical connections to a single entry of the chip unit 16. Hereby, it is possible to apply a simple (and low-cost) chip unit 16 having only a single electrical entry (or few electrical entries). The chip unit 16 and the adapter/controller 18 are electrically connected to the wire member 12 and the coil member 6. However, the coil member 6 does not need to be electrically connected to the adapter/controller 18 if it is electrically connected to the chip unit 16 (as illustrated in FIG. 1(C)).

Four separated apertures 14, 14′ formed as square openings are provided in the sensory layer 3 of the bandage sensory member 2. The apertures 14, 14′ ensure sufficient ventilation to a wound as well as absorption of fluids in case the resulting bandage 5 is used to monitor a wound. The bandage sensory member 2 may in some embodiments also comprise one or more apertures 14, 14′, optionally corresponding to the apertures 14, 14′ of the sensory layer 3.

FIG. 1(C) illustrates a schematic top view of a bandage sensory member 2 according to the invention. Additional layers 4 are not shown. Whereas the bandage sensory members 2 shown in FIG. 1(A) and in FIG. 1(B) have a square periphery, the bandage sensory member 2 illustrated in FIG. 1(C) has a rectangular periphery. The bandage sensory member 2 comprises a sensory layer 3 comprising ten peripherally arranged temperature sensors 8 arranged along the periphery of the sensory layer 3. The ten peripheral temperature sensors 8 are electrically connected by an electric wire member 12 and surrounded by a coil member 6. Two centrally arranged temperature sensors 8 (the sensors between the apertures 14, 14′) are electrically connected to the remaining temperature sensors 8 by the wire member 12. The centrally arranged temperature sensors 8 are configured to be arranged to measure the temperature of wounded tissue. The remaining ten peripheral temperature sensors 8 are, however, configured to detect the temperature in the non-wounded tissue surrounding the wounded tissue.

A chip unit 16 is electrically connected to a coil member 6 surrounding the ten peripheral temperature sensors 8. The coil member 6 is configured to generate and deliver electrical energy to the chip unit 16 by means of induction when an external receiver such as a smartphone 20 is brought in proximity to the bandage sensory member 2 or a resulting bandage 5. The chip unit 16 is electrically connected to an adapter/controller 18 that is electrically connected to all temperature sensors 8.

Six separated apertures 14, 14′ formed as square openings are provided in the sensory layer 3 and/or the bandage sensory member 2. The apertures 14, 14′ ensure sufficient ventilation to a wound in case the resulting bandage 5 is used to monitor a wound.

FIG. 1(D) illustrates a schematic top view of a bandage sensory member 2 according to the invention. Additional layers are not shown. The bandage sensory member 2 is rectangular and comprises a rectangular sensory layer 3 comprising ten peripherally arranged temperature sensors 8 arranged along the periphery of the sensory layer 3 like the bandage sensory member shown in FIG. 1(C). The ten peripheral temperature sensors 8 are electrically connected by an electric wire member 12 and surrounded by a coil member 6, and two centrally arranged temperature sensors 8 (between the apertures 14, 14′) are electrically connected to the remaining temperature sensors 8 by the wire member 12. While the centrally arranged temperature sensors 8 are configured to be arranged to measure the temperature of wounded tissue, the remaining ten peripheral temperature sensors 8 are configured to detect the temperature in the non-wounded tissue surrounding the wounded tissue. However, all temperature sensors 8 may detect the temperature of both wounded tissue and non-wounded tissue.

An adapter/controller 18 is electrically connected to the wire member 12. A chip unit 16 is electrically connected to the adapter/controller 18 and a coil member 6 surrounding the ten peripheral temperature sensors 8, 8′, 8″. The adapter/controller 18 is electrically connected to the chip unit 16 in order to connect a plurality of electrical connections to the adapter/controller 18 and combine electrical signals from different electrical connections to a single entry of the chip unit 16. The coil member 6 is configured to generate and deliver electrical energy to the chip unit 16 by means of induction when an external receiver such as a smartphone 20 is brought in proximity to the bandage sensory member 2 or a resulting bandage 5.

Two separated apertures 14, 14′ formed as rectangular openings are provided in the sensory layer 3 and/or the bandage sensory member 2. The apertures 14, 14′ ensure sufficient ventilation to a wound in case a resulting bandage 5 is used to monitor a wound.

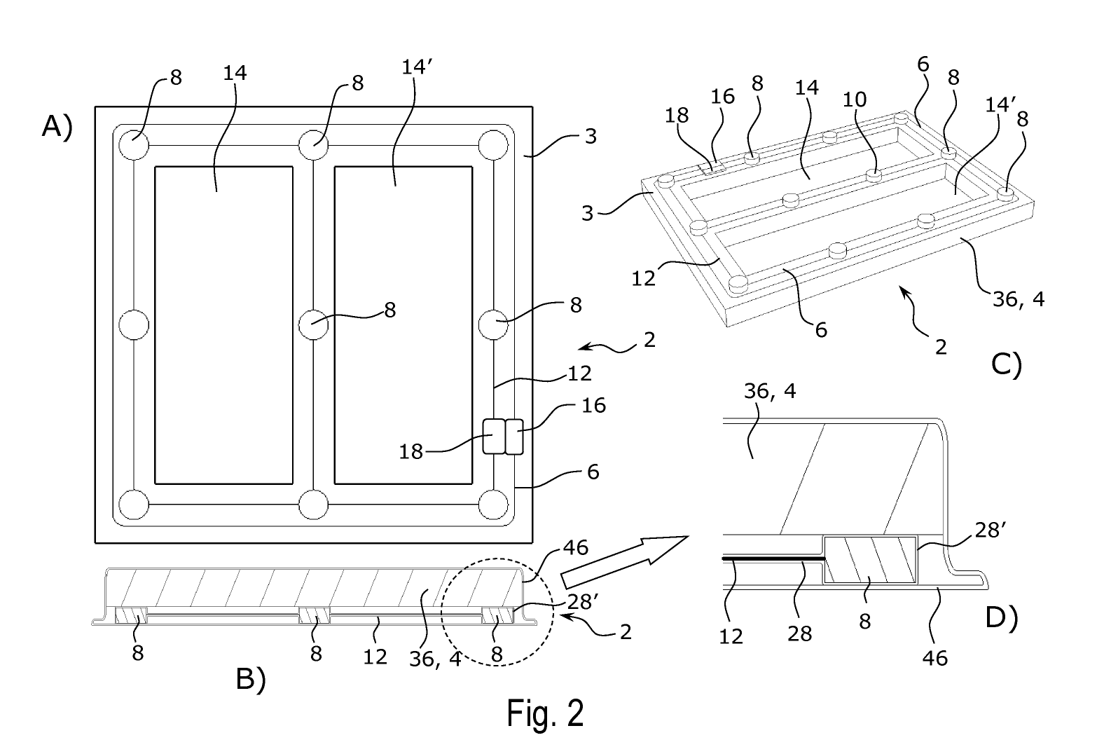

FIG. 2(A) illustrates a schematic top view of a bandage sensory member 2 according to the invention. Additional layers 4 are not shown. The bandage sensory member 2 basically corresponds to the one shown in FIG. 1(A). The bandage sensory member 2 comprises a sensory layer 3 provided with a plurality of first temperature sensors 8 arranged along the square periphery of the sensory layer 3. These temperature sensors 8 are configured to detect the reference temperature of non-wounded tissue.

The sensory layer 3 additionally comprises a centrally arranged additional temperature sensor 8 arranged at the centre of the sensory layer 3 and configured to detect the temperature of wounded tissue. A first aperture 14 and a second aperture 14′ are provided in the sensory layer 3 and/or the bandage sensory member 2. The apertures 14, 14′ ensure sufficient ventilation to a wound as well as absorption of fluids in case a resulting bandage 5 is used to monitor a wound.

The bandage sensory member 2 comprises a chip unit 16 arranged at the sensory layer 3 and electrically connected to an adapter/controller 18 that is electrically connected to the temperature sensors 8 by means of a wire member 12. The chip unit 16 is electrically connected to a coil member 6 surrounding the temperature sensors 8. The coil member 6 is configured to generate and deliver electrical energy to the chip unit 16 by means of induction when an external receiver such as a smartphone 20 is brought in proximity to the bandage sensory member 2 or a resulting bandage 5.

When a smartphone 20 is brought in proximity to the bandage sensory member 2 or a resulting bandage 5, electrical power is generated in the coil member 6, and this electric power is transferred to the chip unit 16 through the coil member 6. The chip unit 16 comprises a processor that reads the temperatures detected by the temperature sensors 8. The chip unit 16 is configured to send the detected temperatures to the smartphone 20 wirelessly by means of the coil member 6.

In one embodiment, the chip unit 16 is configured to calculate the differences between all temperature sensors 8 and detect the maximum temperature difference ΔTₘₐₓ. The chip unit 16 is configured to send the maximum temperature difference ΔTₘₐₓ to the smartphone 20 wirelessly by means of the coil member 6. In another embodiment, the chip unit 16 is configured to calculate only the temperature difference between the sensors 8.

FIG. 2(B) illustrates a cross-sectional view of the bandage sensory member 2 shown in FIG. 2(A). It can be seen that the bandage sensory member 2 comprises an additional layer 4 in the form of an absorbing layer 36 arranged above a plurality of temperature sensors 8 and a wire member 12. The temperature sensors 8 and the wire member 12 are encased by the coating 28, 28′. The coating 28, 28′ is joint to provide a hermetically sealed coating 28, 28′ (another example of an additional layer 4) preventing water and fluid from getting access to the temperature sensors 8 and the wire member 12 that is electrically connecting the temperature sensors 8.

The absorbing layer 36 (example of additional layer 4) and the sensor arrangement (the temperature sensors 8 and the wire member 12) are arranged in a sleeve 46. The bottom surface of the bandage sensory member 2 is intended to be arranged directly on the skin of a person. The sensor arrangement (the temperature sensors 8 and the wire member 12) may be provided as a one-piece member.

FIG. 2(C) illustrates a perspective view of a bandage sensory member 2 according to the invention. The bandage sensory member 2 comprises a sensory layer 3 comprising ten peripherally arranged temperature sensors 8 arranged along the periphery of the sensory layer 3. The ten peripheral temperature sensors 8 are electrically connected by an electric wire member 12 and surrounded by a coil member 6. Two centrally arranged temperature sensors 8 are electrically connected to the remaining temperature sensors 8 by the wire member 12. The centrally arranged temperature sensors 8 are adapted to be arranged in a position in which they can measure the temperature of wounded tissue, while the remaining ten peripheral temperature sensors 8 are configured to be arranged in positions in which they can detect the temperature of the non-wounded tissue surrounding the wounded tissue. All temperature sensors 8 may, however, detect the temperature of both wounded tissue and non-wounded tissue.

The bandage sensory member 2 comprises a chip unit 16 disposed onto the sensory layer 3 and electrically connected to the coil member 6. The chip unit 16 is electrically connected to an adapter/controller 18 that is electrically connected to the wire member 12. The chip unit 16 is configured to receive electrical inputs from the temperature sensors 8 via the adapter/controller 18 when current is induced in the coil member 6.

Two separated apertures 14, 14′ formed as square openings are provided in the sensory layer 3 and/or the bandage sensory member 2. The apertures 14, 14′ ensure sufficient ventilation to a wound in case a resulting bandage 5 is used to monitor a wound.

FIG. 2(D) illustrates a close-up view of a portion of the bandage sensory member 2 shown in FIG. 2(B). The bandage sensory member 2 comprises an absorbing layer 36 (example of an additional layer 4) arranged above a temperature sensor 8 and a wire member 12. The temperature sensor 8 is sealed and encased by a coating 28, whereas the wire member 12 is sealed and encased by the coating 28′. The coating 28, 28′ is joint to provide a hermetically sealed coating 28, 28′ preventing water and fluid from getting access to the temperature sensor 8 and the wire member 12. As apparent, the absorbing layer 36 (additional layer 4) and the sensor arrangement (the sensor 8 and the wire member 12) are provided within a sleeve 46. The sleeve 46 is thinner than both the absorbing layer 36 and the sensor 8.

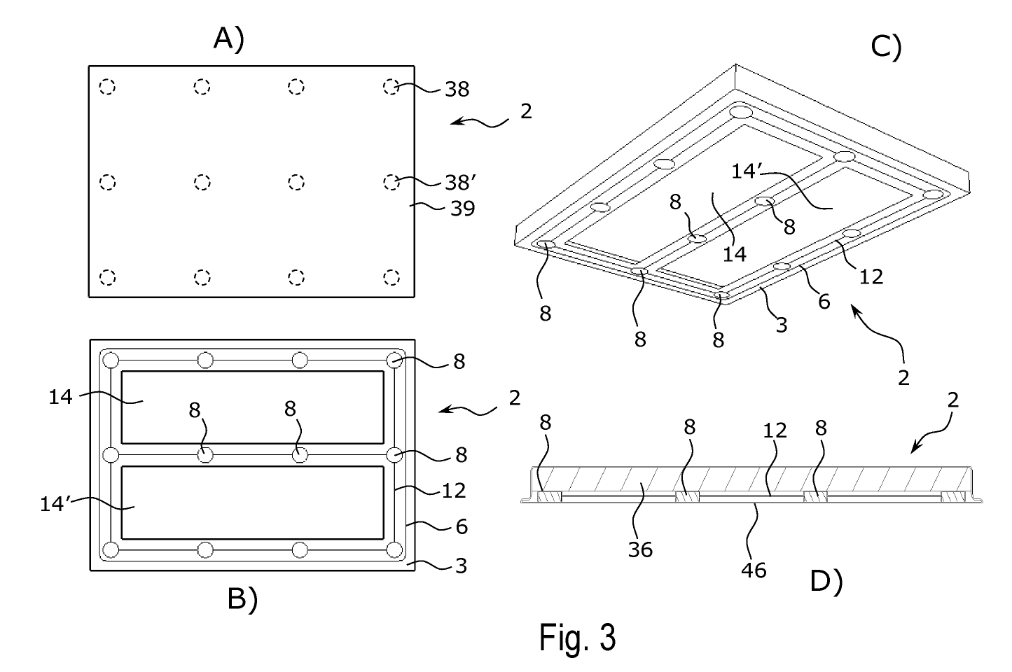

FIG. 3(A) illustrates a schematic top view of a bandage sensory member 2 according to the invention. Seen from the top, the temperature sensors are not visible. However, dotted line indications 38, 38′ are provided at the top surface 39 in order to guide the user when arranging the bandage sensory member 2 or a resulting bandage 5 (the bandage 5 may also be provided with corresponding dotted line indications 38, 38′). Accordingly, the dotted line indications 38, 38′ indicate the positions in which the temperature sensors are provided.

FIG. 3(B) illustrates a bottom view of the bandage sensory member 2 shown in FIG. 3(A). Any additional layers 4 are not shown. The bandage sensory member 2 comprises a rectangular sensory layer 3 comprising ten peripherally arrange temperature sensors 8 arranged along the periphery of the sensory layer 3. The ten peripheral temperature sensors 8 are electrically connected by an electric wire member 12 and surrounded by a coil member 6, and two centrally arranged temperature sensors 8 (between the apertures 14, 14′) are electrically connected to the remaining temperature sensors 8 by the wire member 12. The centrally arranged temperature sensors 8 are configured to be arranged to measure the temperature of wounded tissue. The remaining ten peripheral temperature sensors 8 are configured to detect the temperature of the non-wounded tissue surrounding the wounded tissue.

A chip unit (not shown) is electrically (directly or indirectly via an adapter) connected to the wire member 12 and a coil member 6 surrounding the ten peripheral temperature sensors 8. The coil member 6 is configured to generate and deliver electrical energy to the chip unit by means of induction when an external receiver such as a smartphone 20 is brought in proximity to the bandage sensory member 2 or a resulting bandage 5. Two separated apertures 14, 14′ formed as rectangular openings are provided in the sensory layer 3 and/or the bandage sensory member 2. The apertures 14, 14′ ensure sufficient ventilation to a wound in case a resulting bandage 5 is used to monitor a wound.

FIG. 3(C) illustrates a perspective bottom view of the bandage sensory member 2 shown in FIG. 3(A) and in FIG. 3(B). The bandage sensory member 2 constitutes a flat structure configured to be arranged directly on the skin of a patient. The bandage sensory member 2 is intended to be arranged with the centrally arranged temperature sensors 8 in the wounded tissue, whereas the remaining temperature sensors 8 are configured to be arranged to measure the temperature of non-wounded tissue surrounding the wounded tissue. All temperature sensors 8 can, however, be used to detect the temperature of wounded tissue and non-wounded tissue.

FIG. 3(D) illustrates a cross-sectional view of the bandage sensory member 2 shown in FIG. 3(A), FIG. 3(B) and FIG. 3(C). It can be seen that the bandage sensory member 2 comprises an absorbing layer 36 (example of an additional layer 4) arranged above the temperature sensors 8 and the wire member 12 connecting the temperature sensors 8. The absorbing layer 36 as well as the sensor arrangement (the temperature sensors 8 and the wire member 12 connecting the temperature sensors 8, 8′, 8″) are arranged in a sleeve 46. The bottom side of the bandage sensory member 2 is intended to be place on the skin of a person. However, the bandage sensory member 2 may also be incorporated into a bandage 5.

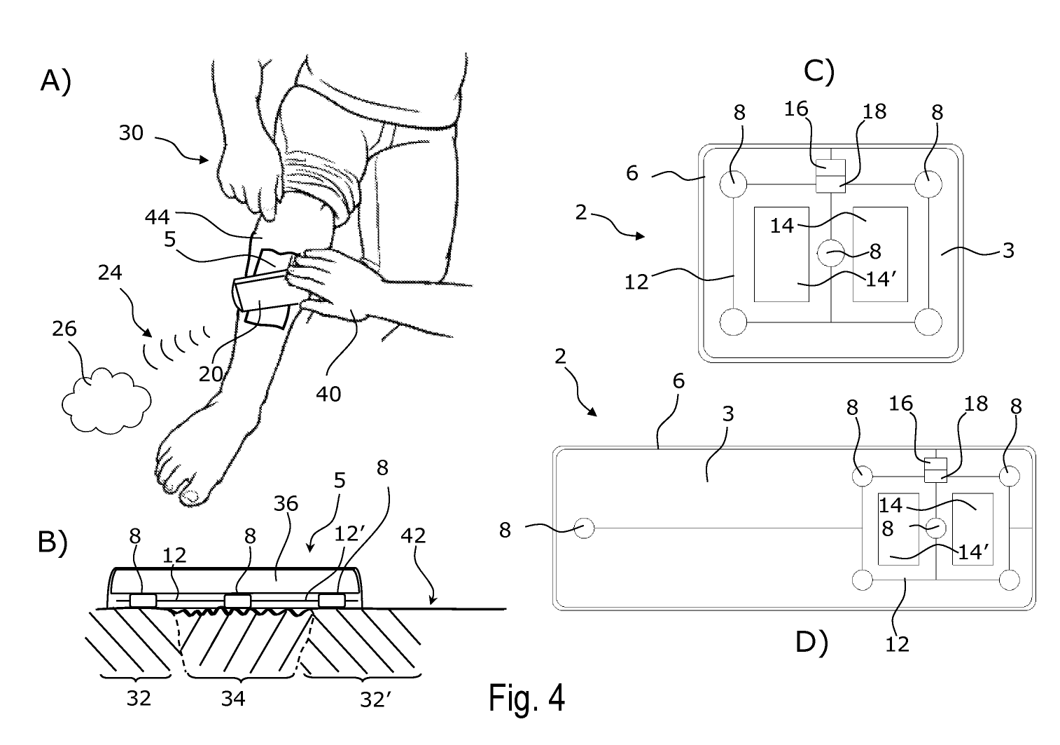

FIG. 4(A) illustrates a perspective view of a patient 30 wearing a bandage 5, and a health care person activating the temperature sensors 8 of the bandage sensory member 2 by means of a smartphone 20. It is important to underline that the patient himself may activate the temperature sensors of the bandage sensory member 2, simply by bringing the smartphone 20 close to (e.g. above) the bandage 5.

The bandage 5 is attached to the patient's 30 leg 44. The health care person holds a smartphone 20 in her hand 40. When bringing the smartphone 20 into a position above the bandage 5 in close distance to the coil member of the bandage sensory member 2, current is generated in the coil member of the bandage sensory member 2. The electrical energy generated is used by the bandage sensory member 2 to perform temperature measurements and to send information to the smartphone 20.

The smartphone 20 can forward information 24 to the Internet 26 so that a database or an external receiver may have access to the measurements made by the temperature sensors 8 of the bandage sensory member 2. Moreover, the smartphone 20 may preferably be configured to generate an alert in case the ΔTₘₐₓ exceeds a predefined value (e.g. 2° C.).

In one embodiment of the invention, the bandage sensory member 2 is configured to detect the highest temperature difference between the temperatures of the temperature sensors 8.

FIG. 4(B) illustrates a cross-sectional view of wounded tissue 34 of a patient. A bandage sensory member 2 is attached to the skin 42 of the patient. This illustrates the embodiment where the bandage sensory member 2 is used without being incorporated into a bandage 5. The bandage sensory member 2 comprises a sensory layer 3 having centrally arranged temperature sensors 8 that is arranged in a position in which they can measure the temperature of the wounded tissue 34. The bandage sensory member 2 comprises a sensory layer 3 with a peripherally arranged temperature sensor 8 and a second peripherally arranged temperature sensor 8 arranged in positions in which they can measure the temperature of the non-wounded tissue 32, 32′ surrounding the wounded tissue 34.

Accordingly, the bandage sensory member 2 can measure the temperatures of the temperature sensors 8. These temperatures can be sent to an external receiver such as a smartphone 20, which can calculate the maximum temperature difference, ΔTₘₐₓ, between the temperatures detected by the temperature sensors 8 and detect if the ΔTₘₐₓ exceeds a predefined value (e.g. 2.5° C.).

In another embodiment of the invention, the temperatures detected by the temperature sensors 8 can be processed by the chip unit 16 in a manner in which the chip unit 16 determines the maximum temperature difference, ΔTₘₐₓ, between the temperatures detected by the temperature sensors 8 and detect if the ΔTₘₐₓ exceeds a predefined value (e.g. 2.5° C.). Accordingly, both the detected temperatures as well as the maximum temperature difference, ΔTₘₐₓ, between the temperatures detected by the temperature sensors 8 may be sent wirelessly to the external receiver.

FIG. 4(C) illustrates a top view of a bandage sensory member 2 according to an embodiment of the invention. Additional layers 4 are not shown. The bandage sensory member 2 comprises a sensory layer 3 comprising four peripherally arranged temperature sensors 8 arranged along the periphery of the sensory layer 3. The four peripheral temperature sensors 8 are electrically connected by an electric wire member 12 and surrounded by a coil member 6. A centrally arranged temperature sensor 8 (arranged at the centre of the sensory layer 3) is electrically connected to the remaining temperature sensors 8 by the wire member 12. The centrally arranged temperature sensor 8 is configured to be arranged to measure the temperature of wounded tissue, while the remaining eight peripheral temperature sensors 8 are configured to detect the temperature in the non-wounded tissue surrounding the wounded tissue. However, all temperature sensors 8 are configured to detect the temperature of both wounded tissue and non-wounded tissue. Accordingly, the position of the sensors 8 determines if the sensors 8 measure the temperature of wounded tissue or the temperature of non-wounded tissue.

A chip unit 16 and an adapter/controller 18 are electrically. The adapter/controller 18 makes it possible to connect a plurality of electrical connections to the adapter/controller 18 and combine electrical signals from different electrical connections to a single entry of the chip unit 16. Hereby, it is possible to apply a simple (and low-cost) chip unit 16 having only a single electrical entry. The chip unit 16 and the adapter/controller 18 are electrically connected to the wire member 12 and the coil member 6.

Two separated apertures 14, 14′ formed as square openings are provided in the sensory layer 3 and/or bandage sensory member 2. The apertures 14, 14′ ensure sufficient ventilation to a wound in case the bandage sensory member 2 or a resulting bandage 5 is used to monitor a wound.

FIG. 4(D) illustrates a bandage sensory member 2 according to another embodiment of the invention. Additional layers 4 are not shown. The bandage member 2 comprises a sensory layer 3 comprising four peripheral temperature sensors 8 arranged in a square formation provided in one end of the elongated rectangular bandage sensory member 2. The four temperature sensors 8 are electrically connected by an electric wire member 12 and surrounded by a coil member 6. A centrally arranged temperature sensor 8 (between the apertures 14, 14′) is electrically connected to the remaining temperature sensors 8 by the wire member 12. The central temperature sensor 8 is arranged in the opposite end to the four peripheral temperature sensors 8. The central temperature sensor 8 is configured to be arranged to measure the temperature of wounded tissue, while the remaining eight peripheral temperature sensors 8 are configured to detect the temperature in non-wounded tissue arranged in a distance from the wounded tissue.

A chip unit 16 and an adapter/controller 18 are electrically connected. The adapter/controller 18 makes it possible to connect a plurality of electrical connections to the adapter/controller 18 and combine electrical signals from different electrical connections to a single entry of the chip unit 16. Accordingly, it is possible to apply a simple (and low-cost) chip unit 16 having only a single electrical entry. The chip unit 16 and the adapter/controller 18 are electrically connected to the wire member 12 and the coil member 6.

Two separated apertures 14, 14′ formed as square openings are provided in the sensory layer 3 and/or the bandage sensory member 2. The apertures 14, 14′ ensure sufficient ventilation to a wound in case the bandage sensory member 2 or a resulting bandage 5 is used to monitor a wound.

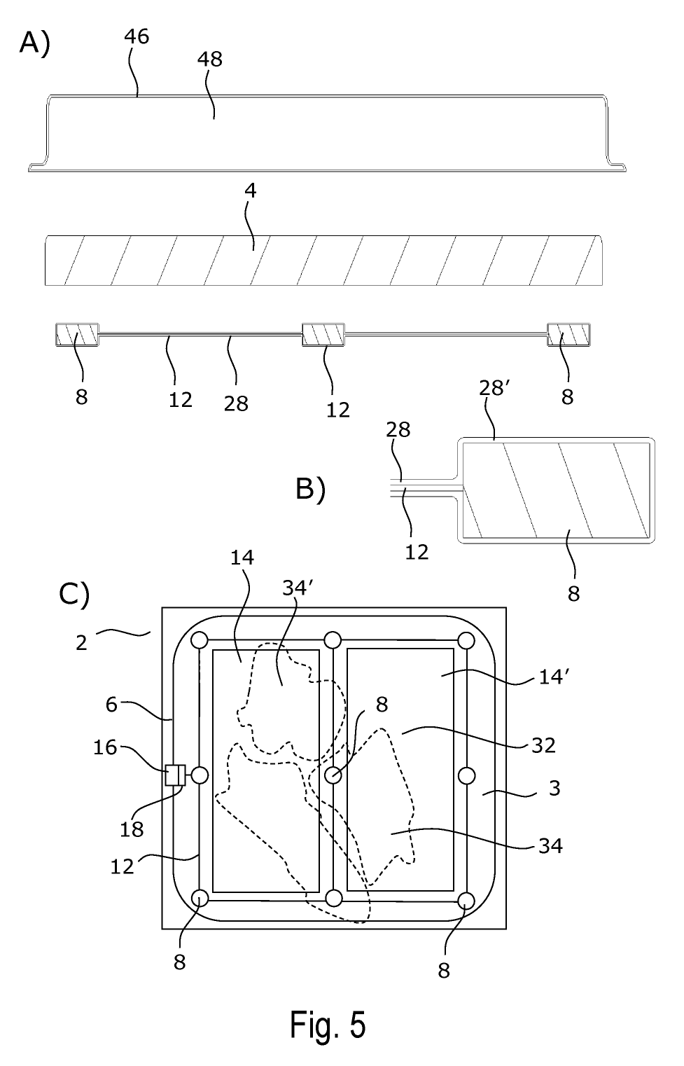

FIG. 5(A) illustrates an exploded view of the bandage sensory member 2 shown in FIG. 2(B). The bandage sensory member 2 comprises a sleeve 46 (an example of an additional layer 4) defining a space 48 adapted to receive an absorbing layer 36 (example of an additional layer 4) and a sensor arrangement consisting of a plurality of temperature sensors 8 and a wire member 12 connecting the temperature sensors 8.

The temperature sensors 8 and the wire member 12 are encased by the coating 28, 28′ (example of additional layer 4). The coating 28, 28′ is joint to provide a hermetically sealed coating 28, 28′ preventing water and fluid from getting access to the temperature sensors 8 and the wire member 12 that is electrically connecting the temperature sensors 8.

The absorbing layer 36 and the sensor arrangement (the temperature sensors 8 and the wire member 12) are configured to be inserted into the sleeve 46 as shown in FIG. 2(B). The sensor arrangement (the temperature sensors 8 and the wire member 12) may be provided as a one-piece member.

FIG. 5(B) illustrates a close-up view of a temperature sensor 8 of the bandage sensory member 2 shown in FIG. 5(A). The temperature sensor 8 has a basically rectangular cross-sectional area and is encased by a coating 28′ (example of additional layer 4). A wire member 12 is electrically connected to the temperature sensor 8′. The wire member 12 is encased by a coating 28. The coating 28, 28′ is joint to provide a hermetically sealed coating 28, 28′ preventing water and fluid from getting access to the temperature sensor 8′ and the wire member 12.

FIG. 5(C) illustrates a schematic top view of a bandage sensory member 2 according to the invention covering wounded tissue 34, 34′. The bandage sensory member 2 comprises a sensory layer 3 provided with a plurality of peripherally arranged temperature sensors 8 arranged along the square periphery of the sensory layer 3. The temperature sensors 8 are configured to detect the reference temperature of non-wounded tissue. The temperature sensors 8 may, however, detect the temperature of any tissue (including wounded tissue).

The sensory layer 3 additionally comprises a centrally arranged additional temperature sensor 8 (at the centre of the sensory layer 3) arranged and configured to detect the temperature of wounded tissue. The central temperature sensor 8 may, however, detect the temperature of any tissue (including non-wounded tissue). A first aperture 14 and a second aperture 14′ are provided in the sensory layer 3 and/or the bandage sensory member 2. The apertures 14, 14′ ensure sufficient ventilation to a wound in case the bandage sensory member 2 or a bandage 5 is used to monitor wounded tissue 34, 34′.

The bandage sensory member 2 comprises a chip unit 16 electrically connected to an adapter/controller 18 that is electrically connected to the temperature sensors 8 by means of a wire member 12. The chip unit 16 is electrically connected to a coil member 6 surrounding the temperature sensors 8. The coil member 6 is configured to generate and deliver electrical energy to the chip unit 16 by means of induction when an external receiver such as a smartphone 20 is brought in proximity to the bandage sensory member 2.

It can be seen that the bandage sensory member 2 covers some first wounded tissue 34 and some second wounded tissue 34′. The central temperature sensor 8 is arranged to detect the temperature of the wounded tissue 34, whereas the peripheral temperature sensors 8 are arranged in a distance from the wounded tissue 34, 34′. Accordingly, the peripheral temperature sensors 8 are arranged and configured to detect the temperature of non-wounded tissue (the reference temperature).

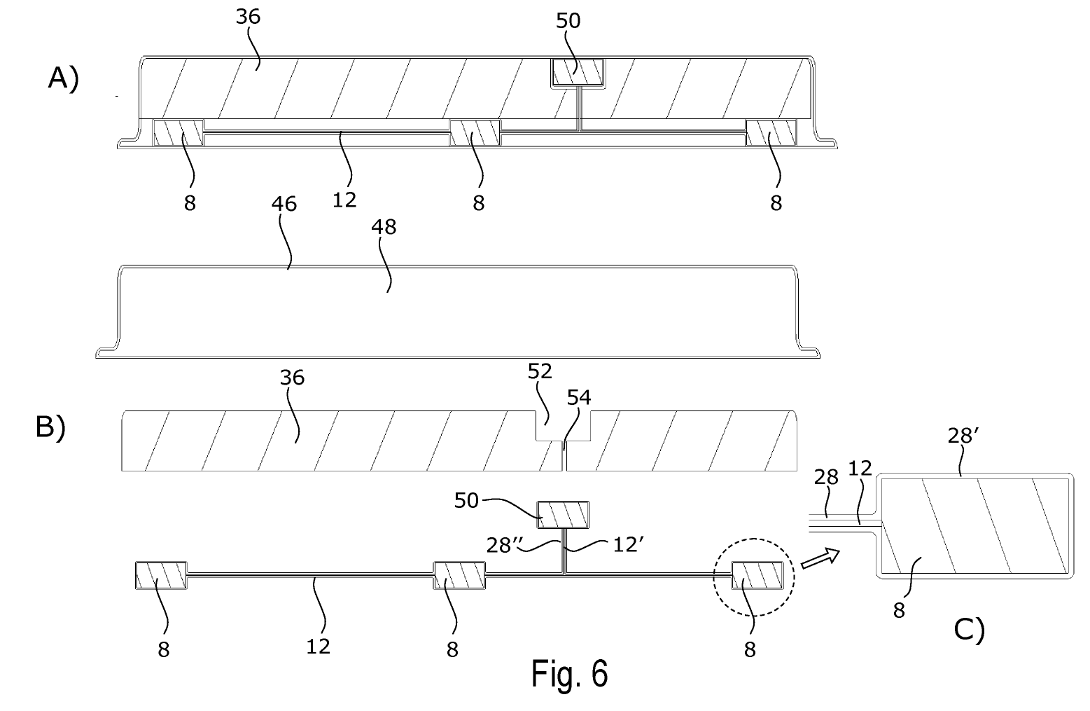

FIG. 6(A) illustrates a cross-sectional view of a bandage sensory member 2 according to the invention. The bandage sensory member 2 comprises an absorbing layer 36 (example of additional layer 4) arranged above a plurality of temperature sensors 8, 50 and a wire member 12 connecting the temperature sensors 8, 50. The temperature sensor 50 is arranged in the additional layer 4 exemplified by the absorbing layer 36. The absorbing layer 36 as well as the sensor arrangement (the temperature sensors 8, 50 and the wire member 12 connecting the temperature sensors 8, 50) are arranged in a sleeve 46 (see FIG. 6(B)). The bottom side of the bandage sensory member 2 is intended to be placed on the skin of a person. However, the bandage sensory member 2 may also be incorporated into a bandage 5.

The bandage sensory member 2 comprises a temperature sensor 50 arranged and configured to detect the ambient temperature. The temperature sensor 50 is arranged in the top portion of the absorbing layer 36.

Hereby, the bandage sensory member 2 may use the ambient temperature to compensate for the ambient temperature. If the ambient temperature is low (e.g. lower than 15° C.), cooling of the bandage sensory member 2 may be expected, and thus lower temperature measurements of the temperature sensors 8 may be achieved.

However, if the ambient temperature is high (e.g. above 30° C.), heating of the bandage sensory member 2 may be expected, and thus higher temperature measurements of the temperature sensors 8 may be achieved. Accordingly, by detecting the ambient temperature, it is possible to adjust for the ambient temperature so that the temperatures detected by the temperature sensors 8 are adjusted according to the ambient temperature. An external receiver (e.g. a smartphone 20) may be configured to adjust the temperatures detected by the temperature sensors 8 on the basis of the detected ambient temperature. The bandage sensory member 2 may comprise a chip unit configured to adjust the temperatures detected by the temperature sensors 8 on the basis of the detected ambient temperature.

FIG. 6(B) illustrates an exploded view of the bandage sensory member 2 shown in FIG. 6(A). It can be seen that the sleeve 46 comprises a space 48 adapted to receive the absorbing layer 36 and the sensor arrangement consisting of a plurality of temperature sensors 8, 50 and the wire members 12, 12′ connecting the temperature sensors 8, 50.

The temperature sensors 8, 50 and the wire members 12, 12′ are encased by a coating 28, 28′. The coating 28, 28′ provides a hermetically sealed coating 28, 28′ preventing water and fluid from getting access to the temperature sensors 8, 50 and the wire member 12, 12′ that is electrically connecting the temperature sensors 8, 50.

The absorbing layer 36 is provided with a recess 52 and a bore 54. The recess 52 is configured to receive and contain the temperature sensor 50, whereas the bore 54 is adapted to contain the wire member 12′.

FIG. 6(C) illustrates a close-up view of a sensor 8 of the bandage sensory member 2 shown in FIG. 6(A) and FIG. 6(B). The temperature sensor 8 has an essentially rectangular cross-section and is encased by a coating 28′. A wire member 12 is electrically connected to the temperature sensor 8. The wire member 12 is encased by a coating 28. The coating 28, 28′ is joint to provide a hermetically sealed coating 28, 28′ preventing water and fluid from getting access to the temperature sensor 8 and the wire member 12.

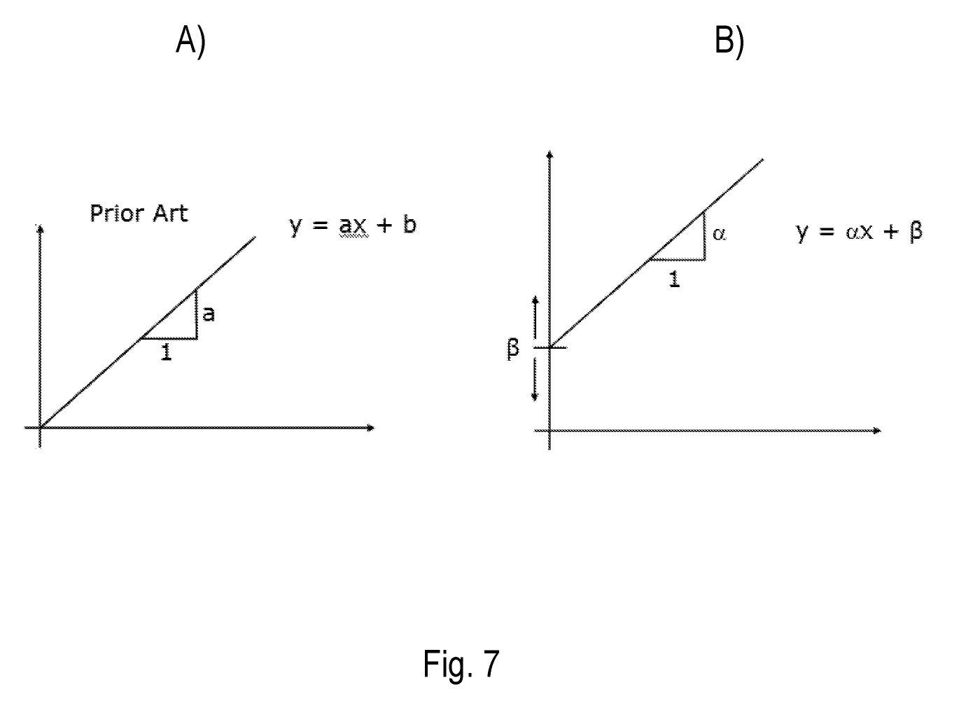

FIG. 7(A) illustrates the calibration curve of the prior art temperature sensors. The sensors are precision sensors capable of measuring the exact and actual temperature. Thus, the prior art temperature sensors are calibrated to a “zero” point actually being 0° C.

FIG. 7(B) illustrates the calibration curve of the temperature sensors used in accordance with the present invention. As can be seen, the temperature sensors may be calibrated to an arbitrary “zero” point (indicated by β). β may be any value as the exact “zero” point needs not be known for application of the temperature sensors in the present invention. Using a suitable application (such as software or an app), the “zero” point β is designated to the temperature sensors.

### LIST OF REFERENCE NUMERALS

2 Bandage sensory member
3 Sensory layer of bandage sensory member
4 Additional layer of bandage sensory member
5 Bandage with bandage sensory member
6 Coil member
8 Temperature sensor(s)
12, 12′ Wire member
14, 14′ Aperture
16 Chip unit
18 Adapter/controller
20 External receiver (e.g. a smartphone)
22 Signal
24 Signal
26 Internet
28, 28′, 28″ Coating
30 Patient
32, 32′ Non-wounded tissue
34, 34′ Wounded tissue
36 Layer (absorbing)
38, 38′ Dotted line
39 Top surface
40 Hand
42 Skin
44 Leg
46 Sleeve
48 Space
50 Temperature sensor(s)
52 Recess
54 Bore
ΔT Temperature difference
ΔTₘₐₓ Maximum temperature difference

1. A bandage sensory member comprising
a sensory layer having a plurality of temperature sensors (8) disposed thereon, and
a chip unit directly or indirectly electrically connected to the temperature sensors,
wherein the chip unit is configured to detect the temperature of each of the temperature sensors and send a wireless signal to an external receiver, wherein the wireless signal contains information about temperature difference (ΔT) and/or maximum temperature difference (ΔTₘₐₓ) between the temperature sensors.

2. The bandage sensory member according to claim 1, characterised in that the bandage sensory member further comprises one or more additional layers.

3. The bandage sensory member according to claim 2, wherein the sensory layer is arranged between two additional layers.

4. The bandage sensory member according to claim 3, characterised in that the sensory layer (3) and the one or more additional layers are joined by heat treatment.

5. The bandage sensory member according to claim 2, characterised in that each of the one or more additional layers is a foam, a textile, a plastic material, or a polymeric material.

6. The bandage sensory member according to claim 2, characterised in that the sensory layer and the one or more additional layers form a plurality of apertures.

7. The bandage sensor member according to claim 1, characterised in that the sensory layer forms a plurality of apertures.

8. The bandage member according to claim 1, characterised in that the sensory layer is a foil, a plastic material, or a paper material.

9. The bandage sensory member according to claim 8, characterised in that the sensory layer further includes an adhesive.

10. The bandage sensory member according to claim 1, characterised in that the temperature sensors (8) are analogue temperature sensors.

11. The bandage sensory member according to claim 1, characterised in that a coil member is integrated in the bandage sensory member, wherein the coil member is configured to generate electrical power by means of induction upon interaction with the external receiver arranged in proximity to the bandage sensory member, wherein the coil member is configured to send the wireless signal to the external receiver.

12. The bandage sensory member according to claim 11, characterised in that the coil member surrounds the temperature sensors.

13. The bandage sensory member according to claim 1, characterised in that the bandage sensory member comprises a temperature sensor arranged and configured to detect the ambient temperature.

14. The bandage sensory member according to claim 1, characterised in that the chip unit is a radio-frequency identification (RFID) chip unit, wherein an adapter/controller is electrically connected to the RFID chip unit and adapted to receive a plurality of electrical connections and combine electrical signals from these different electrical connections to a single entry of the RFID chip unit.

15. The bandage sensory member according to claim 1, characterised in that the chip unit is configured to generate an alert upon occurrence of one or both of (a) no scanning of the temperature sensors has been conducted within a predefined time period (e.g. two or eight hours), and (b) the maximum temperature difference (ΔTₘₐₓ) between the temperature sensors exceeds a predefined value.

16. The bandage sensory member according to claim 1, characterised in that all temperature sensors are electrically connected.

17. The bandage sensory member according to claim 1, characterised in that all temperature sensors are arranged in a matrix configuration.

18. A bandage, characterised in that it comprises a bandage sensory member according to claim 1.
